(19) United States
(12) Patent Application Publication
(10) Pub. No.: US 2003/0078224 A1
(43) Pub. Date: Apr. 24, 2003

(54) Gene expression directed by a super-PSA promoter

(76) Inventors: Leland W.K. Chung, Atlanta, GA (US); Fan Yeung, Charlottesville, VA (US); Chinghai Kao, Indianapolis, IN (US)

Correspondence Address: PENNIE & EDMONDS LLP, 1667 K STREET NW, SUITE 1000, WASHINGTON, DC, 20006

(21) Appl. No.: 10/128,670

(22) Filed: Apr. 24, 2002

Related U.S. Application Data
(63) Continuation of application No. PCT/USOO/29581, filed on Oct. 26, 2000.
(60) Provisional application No. 60/162,223, filed on Oct. 29, 1999.

(51) Int. Cl. A61K 48/00; C12Q 1/68; A01K 67/00; C07H 21/04; C12P 21/02; C12N 5/06

(52) U.S. Cl. 514/44; 800/8; 435/6; 435/69.3; 435/320.1; 435/325; 536/23.2

(57) ABSTRACT

The present invention provides methods and compositions for the delivery and expression of therapeutic genes for treating prostate and non-prostate tumors in a gene therapy setting with therapeutic genes driven by a super PSA promoter. This approach enhances the capability of increasing the size of therapeutic gene inserts and maintaining specificity and efficiency of genes expression. This form of gene therapy strategy can be applied either alone or in combination with other adjuvant therapies or used in combination with various gene therapy strategies to achieve the maximum effect in cancer treatment, and in normal and benign tissues to enhance therapeutic gains.

### CROSS-REFERENCE TO RELATED APPLICATIONS

This application claims the benefit under 35 U.S.C. section 119(e) of co-pending U.S. provisional application No. 60/162,223, filed Oct. 29, 1999, the entire text of which is herein incorporated by reference without disclaimer.

### BACKGROUND

Prostate Specific antigen (PSA) is a popular tumor marker for prostate cancer. It is synthesized and secreted mostly by the luminal epithelial cells of the human prostate (Cleutijens K B J M, Korput H A G M, et al. Mol Endocrin 11 (9): 1256-65). It has been reported that the serum level of PSA is well proportional to tumor size and related to the clinical stages of the disease (Gleave M E. Hsieh J T, et al. J. Urol. 147:1151-59). During hormonal therapy, most patients show an initial often dramatic decrease in PSA levels; however as the disease progress to hormone refractory stage, some of the patients experience a rebound of the PSA level (Montgomery B T, Young C Y, et al. (1992) The prostate 21:63-7). Once initial hormonal therapy has failed, median survival is only 6 months (Montgomery B T, Young C Y, et al. (1992) The prostate 21:63-7). Currently, it is poorly understood how prostate cancer progresses from an androgen dependent (AD) to an androgen independent (Al) stage. The rebound of PSA in hormone refractory prostate cancer cells and its tissue-specific expression pattern makes the PSA promoter a choice candidate for delivering therapeutic gene to prostate cancer cells.

The regulation of PSA expression in hormone-dependent and -independent prostate cancer cells is an interesting focal point. The aim of the study is to understand how hormone-dependent and independent prostate cancer cells upregulate PSA in the presence and absence of androgen stimulation respectively. The in-vitro PSA expression system used in this study consists of two cell lines: LNCaP & C4-2 (Thalmaun G N, Anizinis P E, et al. (1994) Cancer Res. 54:2577-81); (Gleave M E. Hsieh J T, et al. (1992) J. Urol. 147:1151-59). Both cell lines are prostate carcinoma cells that expressed endogenous PSA and androgen receptor (AR). LNCaP represents the early stage of prostate cancer as it is hormone dependent and non-metastatic, while C4-2 represents the advanced stage of prostate cancer as it is hormone independent and highly metastatic (Thalmaun G N, Anizinis P E, et al. (1994) Cancer Res. 54:2577-81); (Montgomery B T, Young C Y, et al. (1992) The prostate 21:63-7). Unlike LNCAP, C4-2 is capable of secreting a large amount of PSA in the absence of androgen (Hsieh J T, Wu H C, et al. (1993) Cancer Res 52:2852-57). One important aspect of this study is to define the regulation of PSA expression in hormone refractory C4-2 cells. The PSA promoter was inserted upstream to a luciferase reporter gene and transiently transfected into LNCaP and C4-2 cells. The promoter of PSA is about 6 Kb long; it contains multiple regulatory elements such as androgen-responsive element (ARE) and prostate-specific enhancer (PSE), and it is tightly regulated by androgen (Schur E R. Henderson G A, et al. JBC 271(12):7043-51 (1996); Rieginan P H J, Vlietstra R J, et al. (1991) Mol Endocrin 5(12):1921-30; Cleutijens K B J M, Korput H A G M, et al. Mol Endocrin (1997) 11:148-161). Promoter analysis studies were done to identify the elements within the promoter that is critical for the expression of PSA in the absence of androgen. Two distinct elements were found to contribute greatly to the PSA promoter activity in hormone refractory C4-2 cells. When these two elements were put together, it surpasses the native PSA promoter activity by 2-4 fold, yet still retains the tissue specificity of the native PSA promoter. The ultimate aim of the study is to identify the transcription factors that interact with these two regulatory elements.

### SUMMARY OF THE INVENTION

Previously, it has been shown that the 632 bp proximal PSA promoter could not direct prostate specificity in transgenic mice, only the full length PSA promoter exhibits tissue specificity in vivo (Schur E R. Henderson G A, et al. (1996) JBC 271(12):7043-51). Full length PSA promoter has also been used for the delivery and expression of therapeutic genes in prostate cancer cells and tumor xenografts. Obviously, certain specific elements in the PSA promoter control the prostate specificity of gene expression. By manipulating the native PSA promoter, Henderson's group have generated a smaller version of PSA promoter PSE for gene therapy purpose (Yeh S, Chang C. (1996) PNAS 93:5517-21). PSE contains the proximal 200 bp PSA promoter and the 440 bp ARE enhancer core (AREc) element that was identified by Trapman (Stamey T A, Yang N, et al. (1989) J. Urol. 141:1088-90). However, PSE is not prostate tissue specific (unpublished data) and its activity is somewhat lower than the native PSA promoter.

A super-PSA promoter was created which has 2-4 fold higher basal activity than the native PSA promoter. Moreover, the super-PSA promoter is also highly inducible by androgen in a prostate tissue-specific manner. This promoter is generated by juxtaposing the ARE enhance core with a newly discovered 120 bp element pTATA. The basal activity of the super PSA promoter is especially impressive in prostate cells that express AR (such as LNCaP, C4-2 & NbE). In cells with neither AR nor PSA expression (such as PC-3), super PSA promoter activity is still 2-4 fold higher than the native PSA promoter. When comparing the super PSA promoter activity between AD LNCaP and Al C4-2, C4-2 clearly could activate the super PSA. promoter to a much higher level than LNCaP in the absence of androgen stimulation. Thus, super PSA promoter is a strong promoter to drive gene expression in hormone refractory tumor cells. The high basal activity of the super PSA promoter together with its great inducibility by androgen make super PSA promoter a highly efficient promoter to drive genes expression both in the absence and presence of androgen in cells.

Additionally, the size of the super-PSA promoter (560 bp) is significantly smaller than the native PSA promoter (6 Kb), which provides advantages for transgenic studies, gene therapy applications and identification of cis-acting and tissue-specific transcription factors for conferring AR-mediated action.

Within the pTATA element, a P2 region was discovered that is essential for the pTATA activity. DNA footprinting was done with the 120 bp pTATA, 2 distinct sites were protected by protein factor(s) from DnaseI digestion. P2 was then identified as one of the sites. It has been suggested that a transcription factor or factors specific for a PSA producing cell interacts with P2 site and activates the promoter activity. This P2 region is of high potential, for it could be used either alone or in conjunction with other promoter elements to generate chimeric promoter constructs for the delivery and expression of genes in normal and tumor cells with high degree of specificity and efficiency. Moreover, by identifying a specific transcription factor that interacts with the P2 site, it could allow the expression of genes in cells without AR nor expressing PSA (such as PC-3). Data is provided to show that when 3 copies of P2 inserted upstream of an artificial TATA box, it could drive high expression of the luciferase reporter gene. The activity of this 3 (P2) construct actually surpass the wild type PSA promoter activity in LNCaP cells. Thus, multiple copies of P2 works well in prostate cells with endogenous AR and PSA. After identifying the cis-acting factor(s) of P2, then a P2-containing promoter together with the cis-acting factor could then be delivered into any cells (including cells that do not produce PSA) to trigger positive regulatory responses.

This concept of specific interaction between a transcription factor and a P2 site or P2-like sequences could exist in another region around the androgen response element III (AREIII) within AREc. It is believed that prostate specific factor(s) interacts with AR binds to specific DNA sequences (e.g. P2 or P2-like consensus sequences), and transactivates the gene expression in PSA producing cells. Therefore, the regions around AREIII could also be used with other promoter elements to generate chimeric constructs that could be used to direct gene expression in a ligand-dependant or -independent and tissue-specific manner.

### BRIEF DESCRIPTION OF THE DRAWINGS

FIG. 1 Summary of Promoter analysis studies.

FIG. 2A comparison of the super PSA promoter (AREc/pTATA) activity with the native PSA promoter (p61) and the PSE promoter (AREc/(PA8).

FIG. 3 The complete sequence of the super-PS A promoter.

FIG. 4 The binding of the potential protein factors to the radiolabeled P2 element in an Electrophoretic˜Mobility-Shift-Assay (EMSA).

FIG. 5 Determination of the basal activity of the Super-PSA promoter and the native PSA promoter in other prostate cell lines.

FIG. 6 Androgen inducibility of the native and super PSA promoter in NbE & PC-3 cells.

### DETAILED DESCRIPTION OF THE INVENTION

PSA is a single chain glycoprotein with a molecular weight of approximately 33 kDa. It is a member of the family of human kallikrein-like serine proteases (Landwall A. (1989). Biochem. Biophys. Res. Commun. 161:1151-59); (Lilja H. (1985). J. Clin. Invest. 76:1899-1903). PSA is synthesized in and secreted by the luminal epithelial cells of the human prostate. In vivo, PSA may function to liquify the seminal coagulum by proteolysis of seminogelin and fibronectin (Cleutijens K B J M, Korput H A G M, et al. Mol Endocrin 11 (Stamey T A, Yang N, et al. J. Urol. 141:1088-90): 1256-65). The PSA gene consists of five exons, and spans a region of approximately 5 Kb (Nazarth L V, Wigel N L. (1996). JBC 271:19900-19907). The gene is clustered with the glandular kallikrein (hGK-1) and renal kallikrein (KLK-1) genes, in an area of 60 Kb on human chromosome 19q13.2-13.4 (Nazarth L V, Wigel N L. (1996). JBC 271:19900-19907). PSA is almost exclusively expressed in the human prostate and prostate derived tumors and tumor cell lines. Gleave M E. Hsieh J T, et al. (1992) J. Urol. 147:1151-59. Therefore, PSA is the mostly widely used serum marker for the diagnosis and management of prostate cancer. Among the gene products specifically expressed by the human prostate, so far only the transcription regulation of the PSA gene has been studied (Schur E R. Henderson G A, et al. JBC 271(12):7043-51. 1996); (Rieginan P H J, Vlietstra R J, et al. (1991) Mol Endocrin 5(12): 1921-30); (Cleutijens K B J M, Korput H A G M, et al. Mol Endocrin (1997) 11:148-161). PSA promoter is about 6 Kb long, it is tightly regulated by androgen through the three androgen responsive elements (AREs) in the promoter (Rieginan P H J, Vlietstra R J, et al. (1991) Mol Endocrin 5(12): 1921-30); (Cleutijens K B J M, Korput H A G M, et al. Mol Endocrin (1997)11:148-161). Upon binding to androgen, ARs translocate into the nucleus and bind to the AREs which will then activate the PSA gene expression. Two of AREs are located in the proximal PSA promoter at positions −170 (ARE I:AGAACAgcaAGTGCT) and −394 (ARE II: GGATCAgggAGTCTC). The third ARE-ARE III is located at ˜4200 bp upstream of the transcription start site. All three ARES contribute to the maximal androgen inducibility of the PSA promoter. ARE III was shown to be part of a very strong androgen regulated enhancer region (AREc). In Western countries, adenocarcinoma of the prostate is the most frequently diagnosed tumor in men, and one of the leading causes of male cancer death (Culig Z, Hobisch A, et al. (1994). Cancer Res. 54:5474-781). Therapeutic approaches for prostate cancer depend on the stage of the malignancy. Initially, the growth of the majority of prostate tumors depends, like normal prostate development, on androgens (Ruiter de PE, Twuwen R. et al. (1995) Mol Cell endocrinol 110:R1-6). Therefore therapy of metastasized tumors is generally based upon androgen ablation or blockade of AR function. After onset of endocrine therapy, most prostate tumors show regression (Stamey T A, Yang N, et al. (1989) J. Urol. 141:1088-90); (Lyss A P. (1987) “Systemic treatment for prostate cancer.” American J Med 83:1120-27). However, essentially all originally hormone responsive tumors become apparently hormone-independent during time (Thalmaun G N, Anizinis P E, et al. (1994) Cancer Res. 54:2577-81). PSA is expressed in the vast majority of prostate cancers, increase of serum PSA during endocrine therapy is generally considered as evidence for recurrence or progression of prostate tumor (Thalmaun G N, Anizinis P E, et al. (1994) Cancer Res. 54:2577-81). The upregulation of PSA in hormone-independent tumor cells may be due to ligand-independent activation of the AR. In prostate cancer cells, Culig et al (Kleinerman D I Troncoso P, et al. (1996). AUA ninety-first annual meeting, Orlando, J. Urol) has shown growth factors like IGF-1 and KGF could induce AR mediated, promoter specific transcriptional activation. Furthermore, cross-talk between AR and both the PKA or PKC-signalling pathways has been reported (Nazarth L V, Wigel N L. (1996). JBC 271:19900-19907); (Ruiter de P E, Twuwen R. et al. (1995) Mol Cell endocrinol 110:R1-6). The role of AR in prostate cancer is still controversial. The majority of locally progressive, hormone refractory tumors show high AR expression, although more heterogeneous than in the normal prostate (Landwall A. (1989). Biochem. Biophys. Res. Commun. 161:1151-59). Interestingly, distant prostatic carcinoma metastases in bone express AR and the expression level seems even higher and more homogeneous than in locally recurrent tumor (Landwall A. (1989). Biochem. Biophys. Res. Commun. 161:1151-59). An increased level of AR expression could allow cancer cells to compensate for their growth and survival under androgen-deprived conditions and to continue their proliferation in the presence of a low level of serum testosterone.

The concept of delivery and expression of therapeutic genes to tumor cells through the use of tissue-specific promoters has been well recognized. This approach decreases the adverse effects of the therapeutic genes on normal cells and increases the specificity and efficiency of gene transfer to normal and tumor cells. Due to its highly tissue-specific expression pattern of PSA in normal, hyperpfastic and malignant prostate epithelia researchers have been trying to use the PSA promoter to direct tissue-specific therapeutic gene expression in prostate cancer cells. The obstacles facing the use of native PSA promoter to direct the specific and efficient gene expression in prostate cells are: 1) Full-length PSA promoter alone without the administration of androgen may not be strong enough to drive the expression of down-stream therapeutic genes in prostate cancer cells. 2) PSA promoter is relatively large that could decrease the capability of genes inserted for therapeutic purposes. These obstacles can be overcome by the following strategies which are described in this invention. First, a super PSA promoter has been generated by juxtaposing AREc enhancer element and pTATA element. This super PSA promoter has 2-4 fold higher activity than the native PSA promoter, and it is highly inducible by androgen in a tissue-specific manner. Moreover, the activity of the super PSA promoter is especially strong in hormone-independent prostate cancer cells which makes it a superior promoter in delivering genes into hormone-independent prostate cancer cells. Additionally, through DNA footprinting experiment, a P2 element has been identified that is essential for the PSA promoter activity. The factor(s) that binds to P2 element may be specific to cells which are capable of expressing endogenous PSA. By manipulating the P2 element, either using multiple copies of it or altering the sequence in the site, the PSA promoter or other tissue-specific promoter activity can be enhanced in normal, benign and malignant prostate cells. The assumption is based upon the observation that when 3 copies of P2 element were used to drive the expression of a reporter gene, tremendous activity is observed in the absence of androgen. Therefore, tandem copies of P2 are able to enhance the activity of a promoter in the PSA-positive cells (e.g. LNCaP and C4-2). Since the interaction of P2 element with its cis-acting factor(s) do not require neither androgen nor AR, it is conceivable that a P2 element containing promoter could deliver therapeutic genes effectively even in PSA- and AR-negative prostate cells (e.g. PC-3), with provision that the P2 cis-acting factor is also introduced into the target cells. Even though the cis-acting factor may be specific for PSA producing cells, the use of P2 element in gene therapy is not limited to prostate cells. It is anticipated that by introducing the cis-acting factor together with the P2 site containing construct, it would be possible to deliver therapeutic genes in any kind of cells. Therefore, by identifying the P2 cis-acting factor(s), P2 element would become a very flexible and strong promoter to deliver therapeutic genes in virtually any given cell.

In summary, the present invention provides methods and compositions for the delivery and expression of therapeutic genes for treating prostate and non-prostate tumors in a gene therapy setting with therapeutic genes driven by a super PSA promoter. It is anticipated that the pTATA and P2 box alone also direct therapeutic gene expression in prostate and non-prostate cells for therapeutic gains. These approaches enhance the capability of increasing the sizes of therapeutic gene inserts and maintaining specificity and efficiency of genes expression. This form of gene therapy strategy can be applied either alone or in combination with other adjuvant therapies or used in combination with various gene therapy strategies to achieve the maximum effect in cancer treatment, and in normal and benign tissues to enhance therapeutic gains.

1 Additional Embodiments of the Invention

In additional embodiments, the invention disclosed herein provides a model for prostate-specific gene transcription. The invention is based in part on the functional characterization described herein of a super-PSA regulatory region, which is highly inducible by androgen and is expressed in a prostate-specific manner.

The present invention provides compositions and methods for screening compounds that modulate expression within prostate cells. In particular, it provides compositions comprising nucleotides from a PSA promoter, and transcriptionally active fragments thereof, as well as nucleic acids that hybridize under highly stringent and moderately stringent conditions to such nucleotides, that control the expression of nucleic acid coding sequences in a prostate specific manner. Specifically provided are expression vectors comprising the super-PSA regulatory region, and transcriptionally active fragments thereof, operably associated to a heterologous reporter gene, e.g., LacZ, and host cells and transgenic animals containing such vectors. The invention also provides methods for using such vectors, cells and animals for screening candidate molecules for agonists and antagonists of prostate-related disorders. Methods for using molecules and compounds identified by the screening assays for therapeutic treatments also are provided.

For example, and not by way of limitation, a composition comprising a reporter gene is operatively linked to a prostate specific regulatory sequence, herein called the super-PSA regulatory region. The super-PSA driven reporter gene is expressed as a transgene in animals. The transgenic animal, and cells derived from the prostate of such transgenic animal, can be used to screen compounds for candidates useful for modulating prostate-related disorders. Without being bound by any particular theory, such compounds are likely to interfere with the function of trans-acting factors, such as transcription factors, cis-acting elements, such as promoters and enhancers, as well as any class of post-transcriptional, translational or post-translational compounds involved in prostate-related disorders. As such, they are powerful candidates for treatment of such disorders.

In one embodiment, the invention provides methods for high throughput screening of compounds that modulate specific expression of genes within the prostate. In this aspect of the invention, cells from the prostate are removed from the transgenic animal and cultured in vitro. The expression of the reporter gene is used to monitor prostate-specific gene activity. In a specific embodiment, LacZ is the reporter gene. Compounds identified by this method can be tested further for their effect on prostate-related disorders in normal animals.

In another embodiment, the transgenic animal models of the invention can be used for in vivo screening to test the mechanism of action of candidate drugs for their effect on prostate-related disorders. Specifically, the effects of the drugs on prostate-related disorders can be assayed.

In another embodiment, a gene therapy method for treating and/or preventing prostate-related disorders is provided. Super-PSA regulatory sequences are used to drive prostate-specific expression of therapeutic molecules and introduced in the cells of the prostate. The method comprises introducing a super-PSA regulatory sequence operatively associated with a nucleic acid encoding a therapeutic molecule into cells of the prostate. In one embodiment, the invention provides a preventative gene therapy method comprising introducing a super-PSA regulatory sequence operatively associated with a nucleic acid encoding a therapeutic molecule into cells of the prostate to delay and/or prevent a prostate-related disorder. In a specific embodiment, the invention provides a gene therapy method for treatment of cancer or other proliferative disorder, including, but not limited to prostate cancer. The super-PSA regulatory sequence is used to direct the expression of one or more coding sequences specifically in the tumor cells of a patient.

The invention further provides methods for screening for novel transcription factors that modulate the super-PSA regulatory sequence. Such novel transcription factors identified by this method can be used as targets for treating prostate-related disorders.

1.1 Polynucleotides and Nucleic Acids of the Invention

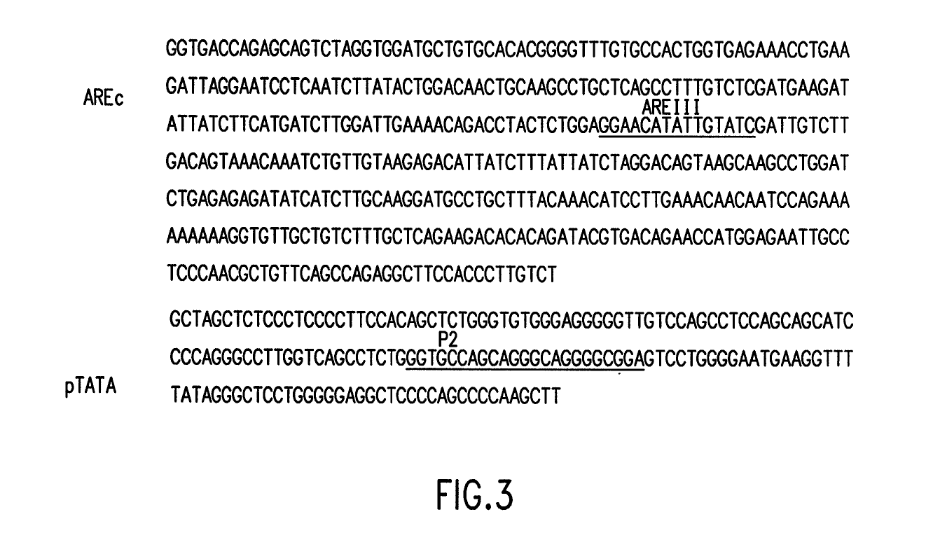

The present invention encompasses polynucleotide sequences comprising 5′ regulatory regions, and transcriptionally active fragments thereof, of the PSA gene. In particular, the present invention provides a polynucleotide comprising the pTATA sequence depicted in FIG. 3 that is located within the PSA gene, and transcriptionally active fragments thereof. The pTATA sequence contains cis elements required to direct prostate-specific transcription in vivo. For example, a 23 bp fragment (P2 region of pTATA) of the pTATA sequence is essential for pTATA activity. The present invention further encompasses a super-PSA regulatory region which comprises the pTATA nucleotide sequence depicted in FIG. 3 juxtaposed to the AREc nucleotide sequence depicted in FIG. 3. This super-PSA regulatory region is sufficient to confer 2-4 fold higher basal activity than the native PSA promoter, and it is highly inducible by androgen in a prostate-specific manner.

The invention further provides probes, primers and fragments of the super-PSA regulatory region. In one embodiment, purified nucleic acids consisting of at least 8 nucleotides (i.e., a hybridizable portion) of a super-PSA regulatory sequence are provided; in other embodiments, the nucleic acids consist of at least 20 (contiguous) nucleotides, 25 nucleotides, 50 nucleotides, 100 nucleotides, 200 nucleotides or 500 nucleotides of a super-PSA sequence. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire regulatory region of a super-PSA sequence.

The probes, primers and fragments of the super-PSA regulatory region provided by the present invention can be used by the research community for various purposes. They can be used as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; and as a probe to “subtract-out” known sequences in the process of discovering other novel polynucleotides. Methods for performing the uses listed above are well known to those skilled in the art. References disclosing such methods include, without limitation, “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The nucleotide sequences of the invention also include nucleotide sequences that have at least 65%, 70%, 75%, 80%, 85%, 90%, 95%, 98% or more nucleotide sequence identity to the nucleotide sequences depicted in FIG. 3, and/or transcriptionally active fragments thereof.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of and a gap penalty of 4 can be used. In an alternate embodiment, alignments can be obtained using the NA_MULTIPLE_ALIGNMENT 1.0 program, using a Gap Weight of 5 and a GapLength Weight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing super-PSA regulatory sequences and/or their complements (i.e., antisense);

(b) DNA expression vectors that contain any of the foregoing super-PSA regulatory element sequences operatively associated with a heterologous gene, such as a reporter gene; and

(c) genetically engineered host cells that contain any of the foregoing super-PSA regulatory element sequences operatively associated with a heterologous gene such that the super-PSA regulatory element directs the expression of the heterologous gene in the host cell.

Also encompassed within the scope of the invention are various transcriptionally active fragments of this regulatory region. A “transcriptionally active” or “transcriptionally functional” fragment of a super-PSA sequence according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the super-PSA regulatory region of the present invention encompass those fragments that are of sufficient length to promote transcription of a heterologous gene, such as a reporter gene, when operatively linked to the super-PSA regulatory sequence and transfected into a prostate cell line. Typically, the regulatory region is placed immediately 5′ to, and is operatively associated with the coding sequence. As used herein, the term “operatively associated” refers to the placement of the regulatory sequence immediately 5′ (upstream) of the reporter gene, such that trans-acting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen may further comprise other nucleotide sequences, either from the PSA gene, or from a heterologous gene. In another embodiment, multiple copies of a promoter sequence, or a fragment thereof, may be linked to each other. For example, the promoter sequence, or a fragment thereof, may be linked to another copy of the promoter sequence, or another fragment thereof, in a head to tail, head to head, or tail to tail orientation. In another embodiment, a prostate cell-specific enhancer may be operatively linked to the super-PSA regulatory sequence, or fragment thereof, and used to enhance transcription from the construct containing the super-PSA regulatory sequence.

Also encompassed within the scope of the invention are modifications of this nucleotide sequence without substantially affecting its transcriptional activities. Such modifications include additions, deletions and substitutions. In addition, any nucleotide sequence that selectively hybridizes to the complement of the sequence depicted in FIG. 3 under stringent conditions, and is capable of activating the expression of a coding sequence is encompassed by the invention. Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & sons, Inc., New York, at p. 2.10.3). Other conditions of high stringency which may be used are well known in the art. In general, for probes between 14 and 70 nucleotides in length the melting temperature (TM) is calculated using the formula: Tm(° C.)=81.5+16.6(log[monovalent cations (molar)])+0.41 (% G+C)-(500/N) where N is the length of the probe. If the hybridization is carried out in a solution containing formamide, the melting temperature is calculated using the equation Tm(° C.)=81.5+16.6(log[monovalent cations (molar)])+0.41(% G+C)-(0.61% formamide)(500/N) where N is the length of the probe. In general, hybridization is carried out at about 20-25 degrees below Tm (for DNA-DNA hybrids) or 10-15 degrees below Tm (for RNADNA hybrids).

The super-PSA regulatory region, or transcriptionally functional fragments thereof, is preferably derived from a mammalian organism. Screening procedures which rely on nucleic acid hybridization make it possible to isolate gene sequences from various organisms. The isolated polynucleotide sequence disclosed herein, or fragments thereof, may be labeled and used to screen a cDNA library constructed from mRNA obtained from appropriate cells or tissues (e.g., prostate tissue) derived from the organism of interest. The hybridization conditions used should be of a lower stringency when the cDNA library is derived from an organism different from the type of organism from which the labeled sequence was derived. Low stringency conditions are well know to those of skill in the art, and will vary depending on the specific organisms from which the library and the labeled sequence are derived. For guidance regarding such conditions see, for example, Sambrook et al., 1989, Molecular Cloning, A Laboratory Manual, Second Edition, Cold Spring Harbor Press, N.Y., and Ausabel et al., 1989, Current Protocols in Molecular Biology, Green Publishing Associates and Wiley Interscience, N.Y., each of which is incorporated herein by reference in its entirety. Further, mammalian super-PSA regulatory region homologues may be isolated from, for example, bovine or other non-human nucleic acid, by performing polymerase chain reaction (PCR) amplification using two primer pools designed on the basis of the nucleotide sequence of the super-PSA regulatory region disclosed herein. The template for the reaction may be cDNA obtained by reverse transcription of the mRNA prepared from, for example, bovine or other non-human cell lines, or tissue known to express the PSA gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the PSA gene may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The super-PSA regulatory regions and transcriptionally functional fragments thereof, and the fragments and probes described herein which serve to identify super-PSA regulatory regions and fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

1.2 Analysis of Prostate-Specific Promoter Activity

The super-PSA regulatory region shows selective tissue and cell-type specificity; i.e., it induces gene expression in a prostate-specific manner. Thus, the regulatory region, and transcriptionally active fragments thereof, of the present invention may be used to induce expression of a heterologous coding sequence in prostate cells. The present invention provides for the use of the super-PSA regulatory region to achieve tissue specific expression of a target gene. The activity and the specificity of the super-PSA regulatory region can further be assessed by monitoring the expression level of a detectable polynucleotide operably associated with the super-PSA regulatory region in different types of cells and tissues. As discussed hereinbelow, the detectable polynucleotide may be either a polynucleotide that specifically hybridizes with a predefined oligonucleotide probe, or a polynucleotide encoding a detectable protein.

1.2.1 Super-PSA Promoter Driven Reporter Constructs

The regulatory polynucleotides according to the invention may be advantageously part of a recombinant expression vector that may be used to express a coding sequence, or reporter gene, in a desired host cell or host organism. The super-PSA regulatory region of the present invention, and transcriptionally active fragments thereof, may be used to direct the expression of a heterologous coding sequence. In particular, the present invention encompasses mammalian super-PSA regulatory regions. In accordance with the present invention, transcriptionally active fragments of the super-PSA regulatory region encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al. 1996, Environ. Microbiol. 62:1133-1140; and Schultz & Yarus, 1990, J. Bacteriol. 172:595-602).

Reporter genes that can be analyzed using calorimetric analysis include, but are not limited to, β-galactosidase (Nolan et al. 1988, Proc. Natl. Acad. Sci. USA 85:2603-07), β-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence which encodes a LacZ gene product, β-galactosidase. The enzyme is very stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli β-glucuronidase gene (GUS) can be used as a reporter gene (Roberts et al. 1989, Curr. Genet. 15:177-180). GUS activity can be detected by various histochemical and fluorogenic substrates, such as X-glucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to super-PSA regulatory region-dependent expression of chloramphenicol resistant cell growth. The use of CAT and the advantages of a selectable reporter gene are well known to those skilled in the art (Eikmanns et al. 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other reporter genes, such as toxic gene products, potentially toxic gene products, and antiproliferation or cytostatic gene products, also can be used. In another embodiment, the detectable reporter polynucleotide may be either a polynucleotide that specifically hybridizes with a predefined oligonucleotide probe, or a polynucleotide encoding a detectable protein, including a PSA polypeptide or a fragment or a variant thereof. This type of assay is well known to those skilled in the art (U.S. Pat. No. 5,502,176 and U.S. Pat. No. 5,266,488).

Super-PSA driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and sequences from a PSA gene. Briefly, the expression of the reporter gene (for example, green fluorescent protein, luciferase, β-galactosidase or chloramphenicol acetyl transferase) is detected when placed under the control of a biologically active polynucleotide fragment. Genomic sequences located upstream of the first exon of the gene may be cloned into any suitable promoter reporter vector. For example, a number of commercially available vectors can be engineered to insert the super-PSA regulatory region of the invention for expression in mammalian host cells. Non-limiting examples of such vectors are pSEAPBasic, pSEAP-Enhancer, pβgal-Basic, pβgal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or β-galactosidase. The regulatory sequences of the PSA gene are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter in the insert.

Expression vectors that comprise a super-PSA regulatory region may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10:169).

1.2.2 Characterization of Transcriptionally Active Regulatory Fragments

A fusion construct comprising a super-PSA regulatory region, or a fragment thereof, can be assayed for transcriptional activity. As a first step in promoter analysis, the transcriptional start point (+1 site) of the prostate-specific gene under study has to be determined using primer extension assay and/or RNAase protection assay, following standard methods (Sambrook et al., 1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for prostate-specific expression. The choice of the type of reporter gene is made based on the application.

In a preferred embodiment, a GFP reporter gene construct is used. The application of green fluorescent protein (GFP) as a reporter is particularly useful in the study of prostate-specific gene promoters. A major advantage of using GFP as a reporter lies in the fact that GFP can be detected in freshly isolated prostate cells without the need for substrates.

In another embodiment of the invention, a Lac Z reporter construct is used. The Lac Z gene product, β-galactosidase, is extremely stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

For promoter analysis in transgenic mice, GFP that has been optimized for expression in mammalian cells is preferred. The promoterless cloning vector pEGFP1 (Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6: 315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407: 313). In an alternate embodiment, transgenic mice containing transgenes with a super-PSA regulatory region upstream of the Lac Z or luciferase reporter genes are utilized.

Putative promoter fragments can be prepared (usually from a parent phage clone containing 8-10 kb genomic DNA including the promoter region) for cloning using methods known in the art. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The promoter fragments, generated by either method, are then ligated into the multiple cloning site of the reporter vector following standard cloning procedures (Sambrook et al., 1989, supra). It is recommended that the DNA sequence of the PCR generated promoter fragments in the constructs be verified prior to generation of transgenic animals. The resulting reporter gene construct will contain the putative promoter fragment located upstream of the reporter gene open reading frame, e.g., GFP or Lac Z cDNA.

In the preferred embodiment, the following protocol is used. Fifty to 100 pg of the reporter gene construct is digested using appropriate restriction endonucleases to release the transgene fragment. The restriction endonuclease cleaved products are resolved in a 1% (w/v) agarose gel containing 0.5 ug/ml ethidium bromide and TAE buffer (1X: 0.04 M Tri-acetate, 0.001 M EDTA, pH 8.0) at 5-6 V/cm. The transgene band is located by size using a UV transilluminator, preferably using long-wavelength UW lamp to reduce nicking of DNA, and the gel piece containing the required band carefully excised. The gel slice and ml of 0.5×TAE buffer is added to a dialysis bag, which has been boiled in 1 mM EDTA, pH 8.0 for 10 minutes (Sambrook et al., 1989, supra) and the ends are fastened. The dialysis bag containing the gel piece is submerged in a horizontal gel electrophoresis chamber containing 0.5×TAE buffer, and electrophoresed at 5-6 V/cm for 45 minutes. The current flow in the electrophoresis chamber is reversed for one minute before stopping the run to release the DNA which may be attached to the wall of the dialysis tube. The TAE buffer containing the electroeluted DNA from the dialysis bag is collected in a fresh eppendorf tube. The gel piece may be observed on the UV transilluminator to ascertain that the electroelution of the DNA is complete.

The electroeluted DNA sample is further purified by passing through Elutip D columns. The matrix of the column is prewashed with 1-2 ml of High salt buffer (1.0 M NaCl, 20 mM Tris. Cl, 1.0 mM EDTA, pH 7.5), followed by a wash with 5 ml of Low salt buffer (0.2 M NaCl, 20 mM Tris. Cl, 1.0 mM EDTA, pH 7.5). A 5 ml syringe is used to apply solutions to the Elutip D column, avoiding reverse flow. The solution containing the electroeluted DNA is loaded slowly. The column is washed with 2-3 ml of Low salt buffer and the DNA is eluted in 0.4 ml of High salt buffer. Two volumes of cold 95% ethanol is added to precipitate DNA. The DNA is collected by centrifugation in a microcentrifuge at 14,000 g for 10 minutes, carefully removing the alcohol without disrupting the DNA pellet. The pellet is washed at least twice with 70% (v/v) ethanol, and dried. The washing and drying steps are important, as residual salt and ethanol are lethal to the developing embryos. The DNA is resuspend in the injection buffer (10 mM TM, 0.1 mM EDTA, pH 7.5 prepared with Milli-Q quality water). The concentration of the purified transgene DNA fragment is determined by measuring the optical density at A₂₆₀ (A₂₆₀=1 for 50 μg/ml DNA) using a spectrophotometer. DNA prepared in this manner is suitable for microinjection into fertilized mouse eggs.

1.2.3 Prostate-Specific Promoter Analysis Using Transgenic Mice

The mammalian super-PSA regulatory region can be used to direct expression of, inter alia, a reporter coding sequence, a homologous gene or a heterologous gene in transgenic animals. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing super-PSA sequences from a different species (e.g., mice expressing super-PSA sequences), as well as animals that have been genetically engineered to over-express endogenous (i.e., same species) super-PSA sequences or animals that have been genetically engineered to knock-out specific sequences.

In one embodiment, the present invention provides for transgenic animals that carry a transgene such as a reporter gene under the control of the super-PSA regulatory region or transcriptionally active fragments thereof in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the super-PSA regulatory region into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, for microinjection of fertilized eggs, a linear DNA fragment (the transgene) containing the regulatory region, the reporter gene and the polyadenylation signals, is excised from the reporter gene construct. The transgene may be gel purified by methods known in the art, for example, by the electroelution method. Following electroelution of gel fragments, any traces of impurities are further removed by passing through Elutip D column (Schleicher & Schuell, Dassel, Germany).

1.3 Screening Assays Compounds that interfere with the abnormal function and/or growth of prostate cells can provide therapies targeting defects in prostate-related disorders. Such compounds may be used to interfere with the onset or the progression of prostate-related disorders. Compounds that stimulate or inhibit promoter activity may be used to ameliorate symptoms of prostate-related disorders.

Transgenic animals or cells containing a super-PSA regulatory region, or fragment thereof, operably linked to a reporter gene, can be used as systems for the screening of agents that modulate super-PSA transcriptional activity. In addition, super-PSA containing transgenic mice provide an experimental model both in vivo and in vitro to develop new methods of treating prostate-related disorders by targeting drugs to cause arrest in the progression of such disorders.

The present invention encompasses screening assays designed to identify compounds that modulate activity of the super-PSA regulatory region. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al., 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Such compounds may further comprise compounds, in particular drugs or members of classes or families of drugs, known to ameliorate the symptoms of a prostate-related disorder.

Such compounds include, but are not limited to, families of antidepressants such as lithium salts, carbamazepine, valproic acid, lysergic acid diethylamide (LSD), p-chlorophenylalanine, p-propyldopacetamide dithiocarbamate derivatives e.g., FLA 63; anti-anxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, a-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and growth factors (e.g., VEGF, FGF, angiopoetins and endostatin).

In one preferred embodiment, primary cultures of germ cells containing a mammalian super-PSA regulatory region operatively linked to a heterologous gene are used to develop assay systems to screen for compounds which can inhibit sequence-specific DNA-protein interactions. Such methods comprise contacting a compound to a cell that expresses a gene under the control of a super-PSA regulatory region, or a transcriptionally active fragment thereof, measuring the level of the gene expression or gene product activity and comparing this level to the level of gene expression or gene product activity produced by the cell in the absence of the compound, such that if the level obtained in the presence of the compound differs from that obtained in its absence, a compound capable of modulating the expression of the mammalian super-PSA regulatory region has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

In another embodiment, microdissection and transillumination can be used. These techniques offer a rapid assay for monitoring effects of putative drugs on prostate cells in transgenic animals containing a super-PSA regulatory region-driven reporter gene. In this embodiment, a test agent is delivered to the transgenic animal by any of a variety of methods. Methods of introducing a test agent may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the prostate cells can be analyzed by the microdissection and transillumination of the prostate cells. If the level of reporter gene expression observed or measured in the presence of the compound differs from that obtained in its absence, a compound capable of modulating the expression of the mammalian super-PSA regulatory region has been identified.

In various embodiments of the invention, compounds that may be used in screens for modulators of prostate-related disorders include peptides, small molecules, both naturally occurring and/or synthetic (e.g., libraries of small molecules or peptides), cell-bound or soluble molecules, organic, non-protein molecules and recombinant molecules that may have super-PSA regulatory region binding capacity and, therefore, may be candidates for pharmaceutical agents.

Alternatively, the proteins and compounds include endogenous cellular components which interact with super-PSA regulatory region sequences in vivo. Cell lysates or tissue homogenates may be screened for proteins or other compounds which bind to the super-PSA regulatory region, or fragment thereof. Such endogenous components may provide new targets for pharmaceutical and therapeutic interventions.

In one embodiment, libraries can be screened. Many libraries are known in the art that can be used, e.g., peptide libraries, chemically synthesized libraries, recombinant (e.g., phage display libraries), and in vitro translation-based libraries. In one embodiment of the present invention, peptide libraries may be used to screen for agonists or antagonists of super-PSA-linked reporter expression. Diversity libraries, such as random or combinatorial peptide or non-peptide libraries can be screened for molecules that specifically modulate super-PSA regulatory region activity. Random peptide libraries consisting of all possible combinations of amino acids attached to a solid phase support may be used to identify peptides that are able to activate or inhibit super-PSA regulatory region activities (Lam, K. S. et al., 1991, Nature 354: 82-84). The screening of peptide libraries may have therapeutic value in the discovery of pharmaceutical agents that stimulate or inhibit the expression of super-PSA by interaction with the promoter region.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) also can be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A specific embodiment of such an in vitro screening assay is described below. The super-PSA regulatory region-reporter vector is used to generate transgenic mice from which primary cultures of super-PSA regulatory region-reporter vector germ cells are established. About 10,000 cells per well are plated in 96-well plates in total volume of 100 μl, using medium appropriate for the cell line. Candidate inhibitors of PSA gene expression are added to the cells. The effect of the inhibitors of PSA gene activation can be determined by measuring the response of the reporter gene driven by the super-PSA regulatory region. This assay could easily be set up in a high-throughput screening mode for evaluation of compound libraries in a 96-well format that reduce (or increase) reporter gene activity, but which are not cytotoxic. After 6 hours of incubation, 100 μl DMEM medium+2.5% fetal bovine serum (FBS) to 1.25% final serum concentration is added to the cells, which are incubated for a total of 24 hours (18 hours more). At 24 hours, the plates are washed with PBS, blot dried, and frozen at −80° C. The plates are thawed the next day and analyzed for the presence of reporter activity.

In a preferred example of an in vivo screening assay, prostate cells derived from transgenic mice can be transplanted into mice with a normal or other desired phenotype (Brinster et al., 1994, Proc. Natl. Acad. Sci. USA 91: 11298-302; Ogawa et al., 1997, Int. J. Dev. Biol. 41:111-12). Such mice can then be used to test the effect of compounds and other various factors on prostate-related disorders. In addition to the compounds and agents listed above, such mice can be used to assay factors or conditions that can be difficult to test using other methods, such as dietary effects, internal pH, temperature, etc.

Once a compound has been identified that inhibits or enhances super-PSA regulatory region activity, it may then be tested in an animal-based assay to determine if the compound exhibits the ability to act as a drug to ameliorate and/or prevent symptoms of a prostate-related disorder, including, but not limited to, prostate cancer.

The assays of the present invention may be first optimized on a small scale (i.e., in test tubes), and then scaled up for high-throughput assays. The screening assays of the present invention maybe performed in vitro, i.e., in test tubes, using purified components or cell lysates. The screening assays of the present invention may also be carried out in intact cells in culture and in animal models. In accordance with the present invention, test compounds which are shown to modulate the activity of the super-PSA regulatory region in vitro, as described herein, will further be assayed in vivo in cultured cells and animal models to determine if the test compound has the similar effects in vivo and to determine the effects of the test compound on prostate-related disorders.

1.4 Compositions and Methods for Therapeutic Use of Super-PSA Nucleotides

Super-PSA polynucleotides, or transcriptionally active fragments thereof, can be used to treat and/or prevent diseases, conditions or disorders that can be ameliorated by modifying the level or the expression of PSA, or a heterologous gene linked to a super-PSA regulatory region, in an prostate-specific manner. Described herein are methods for such therapeutic treatments.

The super-PSA regulatory region may be used to achieve tissue specific expression in gene therapy protocols. In cases where such cells are tumor cells, the induction of a cytotoxic product by the super-PSA regulatory region may be used in the form of cancer gene therapy specifically targeted to prostate tumor cells which contain trans-acting factors required for PSA expression. In this way, the super-PSA regulatory region may serve as a delivery route for a gene therapy approach to cancers involving the prostate. Additionally, antisense, antigene or aptameric oligonucleotides may be delivered to cells using the presently described expression constructs. Ribozymes or single-stranded RNA also can be expressed in a cell to inhibit the expression of a target gene of interest. The target genes for these antisense or ribozyme molecules should be those encoding gene products that are essential for cell maintenance.

The super-PSA regulatory region, and transcriptionally active fragments thereof, of the present invention may be used for a wide variety of purposes, e.g., to down regulate PSA gene expression, or, alternatively, to achieve prostate-specific, stage-specific expression of heterologous genes.

In one embodiment, for example, the endogenous super-PSA regulatory region may be targeted to specifically down-regulate expression of the PSA gene. For example, oligonucleotides complementary to the regulatory region may be designed and delivered to the cells. Such oligonucleotides may anneal to the regulatory sequence and prevent transcription activation. Alternatively, the regulatory sequence, or portions thereof, may be delivered to cells in saturating concentrations to compete for transcription factor binding. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11: 155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In another embodiment, a gene therapy method for ameliorating prostate-related disorders is provided. Super-PSA regulatory region sequences are introduced in the prostate and used to drive prostate-specific expression of drugs or toxins. The method comprises introducing a super-PSA regulatory region sequence operatively associated with a drug or toxin gene into the prostate.

In yet another embodiment, the invention provides a gene therapy method for treatment of cancer or other proliferative disorders. The super-PSA regulatory region is used to direct the expression of one or more proteins specifically in prostate tumor cells of a patient. Such proteins may be, for example, tumor suppressor genes, thymidine kinase (used in combination with acyclovir), toxins or proteins involved in cell killing, such as proteins involved in the apoptosis pathway.

In still another embodiment, the invention provides a preventative gene therapy method for preventing and/or delaying the onset of prostate-related disorders. The super-PSA regulatory region is introduced in the prostate and used to drive prostate-specific expression of therapeutic compounds. The method comprises introducing a super-PSA regulatory region sequence operatively associated with a nucleic acid encoding a therapeutic compound into the prostate to prevent and/or delay the onset of prostate-related disorders.

Methods for introducing genes for expression in mammalian cells are well known in the field. Generally, for such gene therapy methods, the nucleic acid is directly administered in vivo into a target cell or a transgenic mouse that expresses a super-PSA regulatory region operably linked to a reporter gene. This can be accomplished by any method known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g. a gene gun; Biolistic, Dupont), by coating with lipids or cell-surface receptors or transfecting agents, by encapsulation in liposomes, microparticles, or microcapsules, by administering it in linkage to a peptide which is known to enter the nucleus or by administering it in linkage to a ligand subject to receptor-mediated endocytosis (see e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

The oligonucleotide may comprise at least one modified base moiety which is selected from the group including, but not limited to: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Endogenous target gene expression also can be reduced by inactivating or “knocking out” the super-PSA regulatory region using targeted homologous recombination (e.g., see Smithies et al, 1985, Nature 317:230-234; Thomas and Capecchi, 1987, Cell 51:503-512; Thompson et al., 1989, Cell 5:313-321; each of which is incorporated by reference herein in its entirety). For example, a mutant, non-functional target gene (or a completely unrelated DNA sequence) flanked by DNA homologous to the regulatory region of the PSA gene can be used, with or without a selectable marker and/or a negative selectable marker, to transfect cells that express the target gene in vivo. Insertion of the DNA construct, via targeted homologous recombination, results in inactivation of the super-PSA regulatory region. This approach can be adapted for use in humans provided the recombinant DNA constructs are directly administered or targeted to the required site in vivo using appropriate vectors.

In an alternative embodiment, endogenous target gene expression can be reduced by targeting deoxyribonucleotide sequences complementary to the super-PSA regulatory region to form triple helical structures that prevent transcription of the target gene in target cells in the body. (See generally, Helene, 1991, Anticancer Drug Des., 6(6):56920 584; Helene et al., 1992, Ann. N.Y. Acad. Sci., 660:27-36; and Maher, 1992, Bioassays 14(12):807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

In a specific embodiment, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The anti-sense RNA and DNA molecules and triple helix molecules of the invention may be prepared by any method known in the art for the synthesis of nucleic acid molecules. These include techniques for chemically synthesizing oligodeoxyri-bonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phospho-diesterase linkages within the oligodeoxyribonucleotide backbone.

The super-PSA regulatory region, and transcriptionally active fragments thereof, of the present invention can be used to express the PSA gene in an altered manner as compared to expression in a normal cell. The super-PSA regulatory region, and transcriptionally active fragments thereof, of the present invention also can be used to achieve tissue specific expression of a target gene. Thus, it is possible to design appropriate therapeutic and diagnostic techniques directed to this regulatory sequence in order to modulate the expression of a target gene. In accordance with the present invention, the term “modulate” encompasses the suppression or augmentation of expression of a target gene and also encompasses the tissue specific suppression or expression of a target gene. When a cell proliferative disorder is associated with underexpression or overexpression of a PSA gene product, oligonucleotide based compounds such as those described herein, including antisense oligonucleotides, may be used to modulate expression of the PSA gene. For example, where the associated disorder is cancer, the induction of a cytotoxic gene product utilizing the super-PSA regulatory region may be used as a cancer therapy. One of skill in the art can determine if a particular therapeutic course of treatment is successful by several methods known to those of skill in the art, including muscle fiber analysis or biopsy.

1.4.1 Inhibitory, Antisense, Ribozyme and Triple Helix Approaches

In another embodiment, symptoms of disorders involving the prostate may be ameliorated by decreasing the level of super-PSA regulatory region activity by using well-known antisense, gene “knock-out,” ribozyme and/or triple helix methods to decrease the level of super-PSA regulatory region expression. Among the compounds that exhibit the ability to modulate the activity, expression or synthesis of the super-PSA regulatory region, including the ability to ameliorate the symptoms of a prostate-related disorder are antisense, ribozyme and triple helix molecules. Such molecules may be designed to reduce or inhibit either unimpaired, or if appropriate, mutant super-PSA regulatory region activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the gene of interest could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit target gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1 -methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15:6625-6641). The oligonucleotide is a 2′-0-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides maybe synthesized by the method of Stein, et al. (1988, Nucl. Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to the target gene coding region sequence could be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g. antisense linked to peptides or antibodies that specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

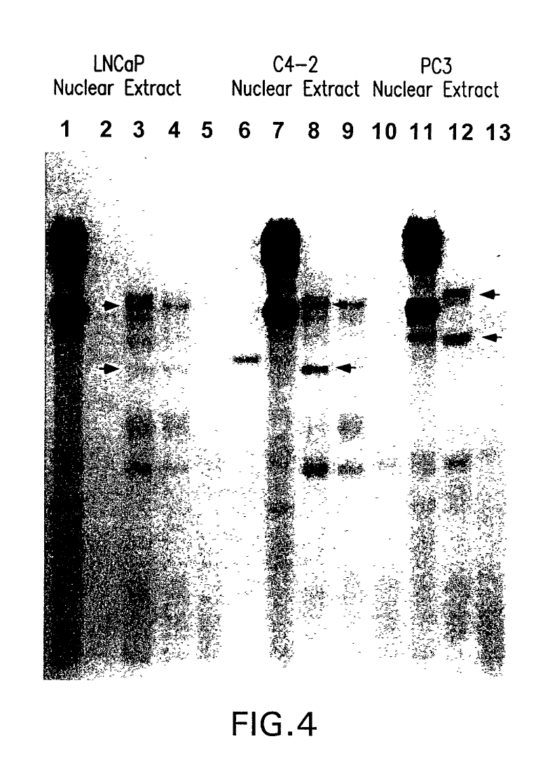

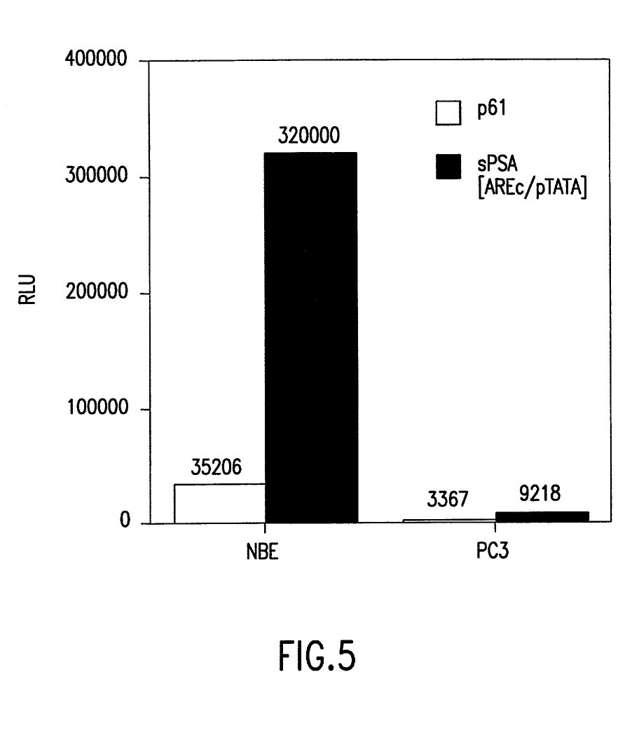

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods, such as those described below, that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

1.4.2 Gene Replacement Therapy

The nucleic acid sequences of the invention, described above, can be utilized for transferring recombinant nucleic acid sequences to cells and expressing said sequences in recipient cells. Such techniques can be used, for example, in marking cells or for the treatment of a prostate-related disorders. Such treatment can be in the form of gene replacement therapy. Specifically, one or more copies of a normal gene or a portion of the gene that directs the production of a gene product exhibiting normal gene function, may be inserted into the appropriate cells within a patient, using vectors that include, but are not limited to adenovirus, adeno-associated virus and retrovirus vectors, in addition to other particles that introduce DNA into cells, such as liposomes.

In one embodiment, techniques for delivery involve direct administration, e.g., by stereotactic delivery of such gene sequences to the site of the cells in which the gene sequences are to be expressed.

Additional methods that may be utilized to increase the overall level of gene expression and/or gene product activity include using targeted homologous recombination methods, as discussed above, to modify the expression characteristics of an endogenous gene in a cell or microorganism by inserting a heterologous DNA regulatory element such that the inserted regulatory element is operatively linked with the endogenous gene in question. Targeted homologous recombination can thus be used to activate transcription of an endogenous gene that is “transcriptionally silent”, i.e., is not normally expressed or is normally expressed at very low levels, or to enhance the expression of an endogenous gene that is normally expressed.

Further, the overall level of target gene expression and/or gene product activity may be increased by the introduction of appropriate target gene-expressing cells, preferably autologous cells, into a patient at positions and in numbers that are sufficient to ameliorate the symptoms of a prostate-related disorder. Such cells may be either recombinant or non-recombinant.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above that are capable of modulating activity of a super-PSA regulatory region can be administered using standard techniques that are well known to those of skill in the art.

1.5 Pharmaceutical Preparations and Methods of Administration

The compounds that are determined to modify super-PSA regulatory region activity or gene product activity can be administered to a patient at therapeutically effective doses to treat or ameliorate a prostate-related disorder. A therapeutically effective dose refers to that amount of the compound sufficient to result in amelioration of symptoms of such a disorder.

1.5.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

1.5.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g. almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds also may be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

2 Example: Promoter Analysis Studies

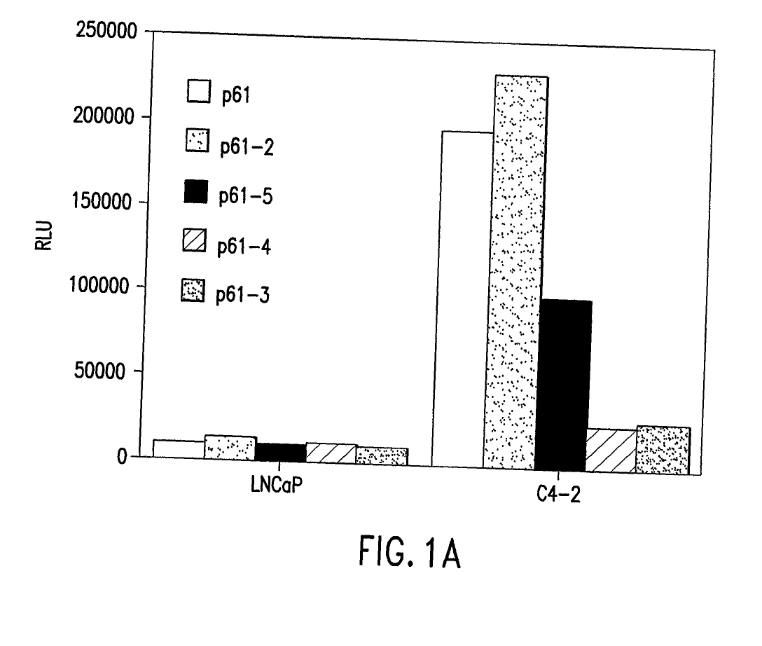

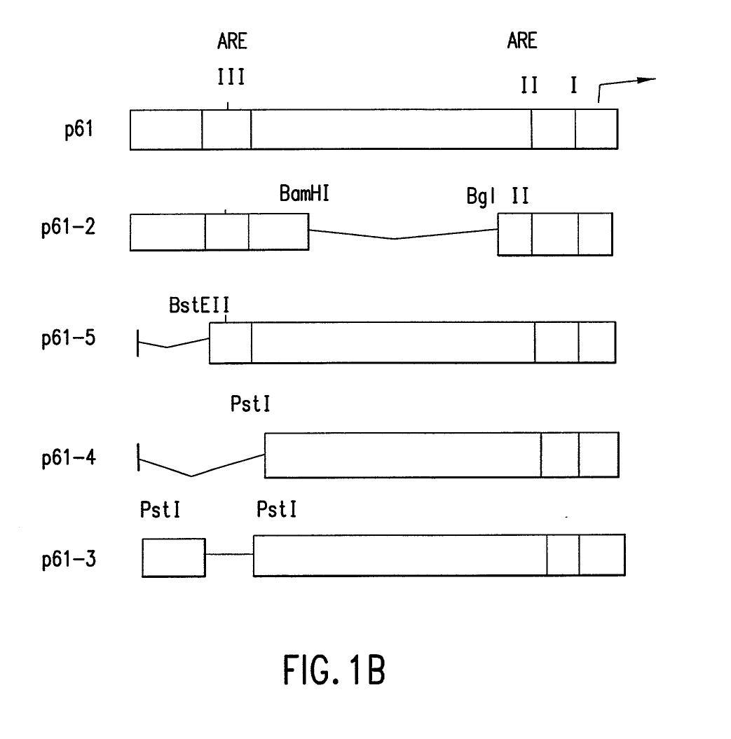

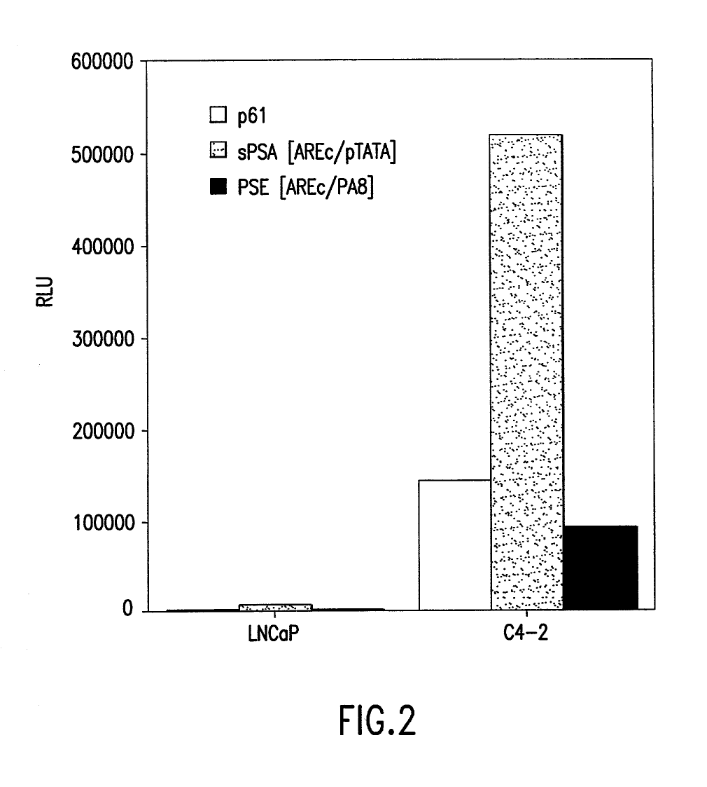

In this example, promoter analysis studies data are presented in FIG. 1. Wild type PSA promoter is depicted as p61, the length of the promoter is ˜6 Kb, the locations of androgen response elements (ARE) I, II & III are shown within the promoter. And the transcription start site (TATA box) is represented by the arrow. The full length promoter was inserted upstream to a luciferase reporter gene and were transiently transfected into LNCaP and C4-2 cells. Relative luciferase unit (RLU) is obtained by normalizing the luciferase activity with the internal control-CMV/beta-gal. In the absence of androgen stimulation, the native PSA promoter is ˜15 fold more active in C4-2 than in LNCaP. Different deletion constructs were generated to identify regions on the promoter that are important for the activation of PSA promoter in the absence of androgen. p61-2 construct contains internal deletion between ARE II & III. This deletion does nor affect the native promoter activity, therefore the deleted region may not be essential for the PSA promoter activity. p61-5 contains terminal deletion, downstream of the ARE III. The activity of this construct drops about 50%. However, as the deletion extend upstream to ARE III, we see a tremendous decline (˜85%) in promoter activity. The additional deletion (500 bp) in p61-4 obviously causes this significant decrease in promoter activity. Therefore, when the 500 bp (AREc) was deleted in the p61-3 construct, it results in a similar activity drops as the p61-4. This strongly suggests that the AREc is essential for the PSA promoter activity in die absence of androgen.

3 Example: Comparison of the Super Psa Promoter (Arec/ptata) Activity with the Native Psa Promoter (P61) and the Pse Promoter (Arec/(Pa8)

A comparison of the super PSA promoter (AREc/pTATA) activity with the native PSA promoter (p61) and the PSE promoter (AREc/(PA8). In addition to AREc, the possible cooperativity between AREI, II and III was tested. A DNA sequence in pTATA region was identified which confers ligand-independent activation of PSA promoter activity in C4-2 cells (see below). The size of the super PSA promoter is only 560 bp which is significantly smaller than the 6 Kb wild type PSA promoter, and it is also smaller than the 1071 bp PSE promoter. Yet, the super PSA promoter has 2-4 fold higher activity when compare to the wild type and ˜5 fold higher activity when comparing with the PSE promoter. Moreover, super PSA promoter is highly inducible in LNCaP (52 fold) and C4-2 (16 fold). The high basal and androgen-induced super PSA promoter activity was observed in cells that expressed AR activity, suggesting that AR and a yet undefined co-factor(s) in C4-2 & LNCaP but not PC3 cells are responsible for upregulating PSA promoter activity (see below). However, the pTATA region alone could also be responsible for upregulating PSA promoter activity in C4-2 cells in an androgen- and AR-independent manner (see below). From this graph, it is clear that super PSA promoter has much higher activities in the AI C4-2 than AD LNCaP cells, this makes it a superior promoter to deliver and express genes in Al prostate cancer cells.

4 Example: Determination of the Complete Sequence of the Super-PSA Promoter

The complete sequence of the super-PSA promoter consists of the ARE enhancer core (AREc) that identified by Trapman (Stamey T A, Yang N, et al. J Urol. 141:1088-90) and a new pTATA element which is identified as described herein. The androgen inducibility resides within the ARE enhancer core region. In addition to the well-characterized androgen responsive element (ARE) III, other AREs have also been identified within the enhancer core recently (Lyss A P. (1987) American J Med 83:1120-27). The androgen inducibility of AREc relies heavily on the AREIII and is specific to cells with endogenous PSA (Stamey T A, Yang N, et al. J. Urol. 141:1088-90). It has been suggested that in the absence of androgen, AR could be signal through a ligand-independent pathway involving selected growth factors and intracellular signal transduction in androgen independent prostate cancer cells (Culig Z, Hobisch A, et al. (1994). Cancer Res. 54:547478); Nazarth L V, Wigel N L. (1996). JBC 271:19900-19907); Ruiter de P E, Twuwen R. et al. (1995) Mol Cell endocrinol 110:R1-6). AR could complexes with cofactor(s) and together could then bind to the AREc and transactivate PSA gene expression (Hsieh J T, Wu H C, et al. (1993) Cancer Res 52:2852-57). It is speculated that the regions surround the AREIII are the binding sites for these protein complexes. Therefore, any alterations of the regions may enhance or diminish the super-PSA promoter activity. The pTATA by itself is a strong element: when juxtaposed to the AREc it synegerizes with the enhancer core to give a superior promoter activity that surpasses the native PSA promoter. Through DNA footprinting experiment, 2 sites in pTATA were identified that are protected by protein factors in C4-2 and LNCAP nuclear extracts. Upon comparison with a sequence marker, the exact location of the protected regions were identified. One of the protected regions was named P2 which was extremely important for pTATA activity. The deletion of P2 eliminates most of the pTATA activity in prostate cancer cells. Data was obtained suggesting that specific transcription factor(s) binds to P2 which may interact with the general transcription machinery to enhance PSA gene expression in the absence of androgen. Furthermore, this interaction does not require neither androgen nor its receptor.

5 Example: Determination of the Binding of The Potential Protein Factors to the Radiolabeled P2 Element

In this example, the binding of the potential protein factors to the radiolabeled P2 element in an Electrophoretic˜Mobility-Shift-Assay (EMSA) was determined. Due to their higher molecular weights, these protein-DNA complexes migrate slower than the free probes in the gel, producing distinct bands corresponds to their own molecular weight. LNCaP nuclear extract was used in lanes 1-5, C4-2 nuclear extract was used in lanes 6-10 and PC3 nuclear extract was used in lanes 11-13. Through consensus sites search, P2 appears to have high homology with SP-1 sites, so SP-1 sites were used to compare with the P2 site. In lanes 1, 7 & 11, SP-1 site gives 3 distinct bands which correspond to the binding of SP 1, SP-2 & SP-3 proteins, and these bands can be competed away specifically with non-labeled SP-I sites (lanes 2, 6). With P2 site, there are several bands detected (lanes 3, 8 & 12). Bands marked with arrows are considered to be major bands and none of them are similar to the bands produced by the SP-1 site. Moreover, non-radiolabeled SP-1 site was unable to compete away the protein-DNA complexes produce by P2 (lanes 4, 9), while non-labeled P2 site can specifically compete away the protein-DNA complexes (lanes 5, 10). Thus, it is reasonable to conclude that the P2 associating factors may not be SP-1 proteins. In addition, the two bands indicated in lanes 3 and 8 are more intense in C4-2 nuclear extract than LNCaP nuclear extract, suggesting there may be an upregulation of the factors in hormone independent C4-2 cells which in turn might enhance the PSA promoter activity in C4-2. With PC3 nuclear extract, P2 element also produces two major hands (lanes 12 & 13), but their sizes are clearly different from the bands of LNCaP (lane 3) and C4-2 (lane 8). It is possible that different: factors occupy the P2 site in PC3 cells but failed to upregulate PSA promoter activity. Therefore, the protein factor(s) associate with P2 site in the pTATA is specific to PSA-producing cell like C4-2 and LNCaP.

6 Example: Determination of the Basal Activity and the Androgen Inducibility of the Super PSA Promoter

In this example, the basal activity and the Androgen inducibility of the native and super PSA promoter were determined. The super PSA promoter consistently has a higher basal activity than the native PSA promoter in other prostate cell lines (FIG. 5). NbE is a spontaneous immortalized normal rat prostate epithelial cell line which comprised a super PSA promoter activity ˜10 fold higher than the native PSA promoter activity. The level of the super PSA promoter activity in NbE is similar to that of the AI C4-2 cells. PC3 is a human prostate cancer cell line. It does not have endogenous AR nor PSA, so the native PSA promoter activity has been conventionally lower. However, the super-PSA promoter is still capable of surpassing the native PSA promoter activity in PC3 by ˜3 fold.

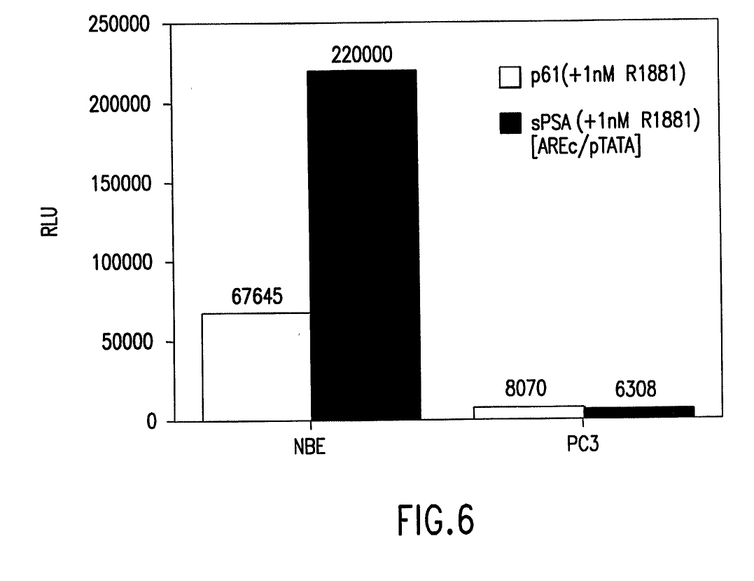

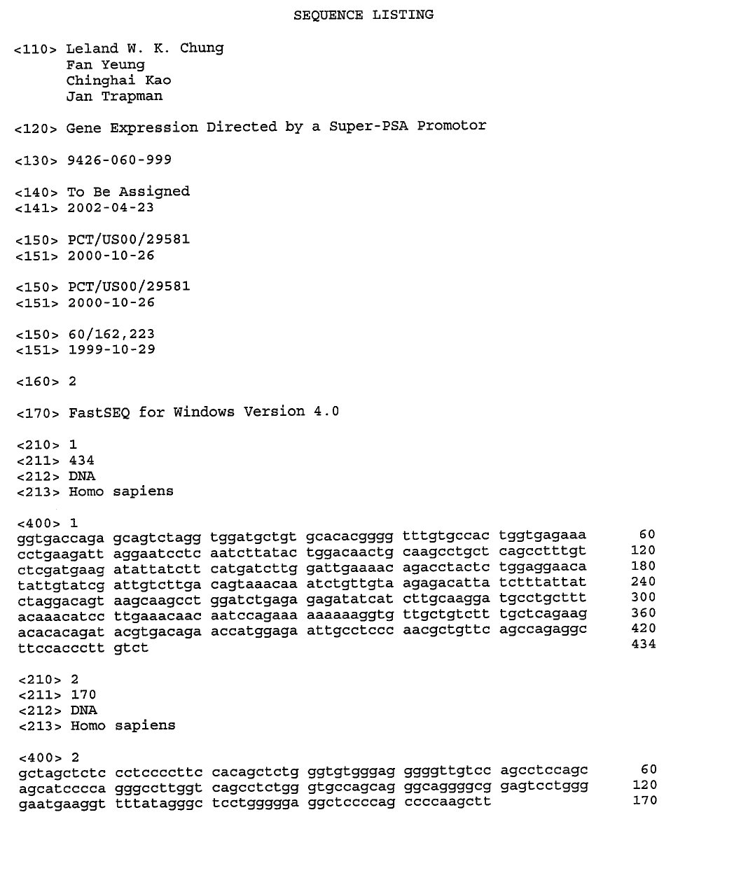

The androgen inducibility of the native and super PSA promoter in NbE & PC-3 cells is shown in FIG. 6. When the androgen inducibility of the native and super PSA promoter in NbE & PC-3 cells from FIG. 6 is compared to FIG. 5, p61 but not AREc/pTATA promoter-mediated reporter activity in NbE and PC-3 cells can be further enhanced by an androgen agonist, R-1881. Conversely, a somewhat reduced AREc/pTATA-mediated reporter activity exhibited in NbE and PC-3 cells subsequent to R-1881 exposure. The exact mechanism of these differential responses at the present time is unclear. It suffices to say, however, that super PSA promoter could drive the expression of target genes in cells that are of prostate origin whether they produce PSA or not. Super PSA promoter, however, is much more active in prostate cells that contain AR (such as C4-2, LNCaP & NbE) than those that do not (such as PC-3).

What is claimed is:

1. An isolated polynucleotide comprising;
(a) the pTATA nucleotide sequence depicted in FIG. 3,
(b) the pTATA nucleotide sequence depicted in FIG. 3 juxtaposed to the AREc nucleotide sequence depicted in FIG. 3, or
(c) a transcriptionally active fragment of the isolated polynucleotide of (a) or (b).

2. An isolated polynucleotide comprising the P2 nucleotide sequence depicted in FIG. 3, or a transcriptionally active fragment thereof.

3. An isolated polynucleotide that hybridizes under highly stringent conditions to the complement of the polynucleotide of claim 1.

4. An isolated polynucleotide that hybridizes under moderately stringent conditions to the complement of the polynucleotide of claim 1.

5. An isolated polynucleotide that comprises the complement of the polynucleotide of claim 1.

6. An isolated polynucleotide comprising the polynucleotide of claims 1 or 2 operably associated with a heterologous coding sequence.

7. A vector comprising the polynucleotide of claims 1, 2, 3 or 4.

8. An expression vector comprising the polynucleotide of claims 1, 2, 3 or 4 operably associated with a heterologous coding sequence.

9. A genetically engineered host cell comprising the polynucleotide of claims 1, 2, 3 or 4.

10. A genetically engineered host cell comprising the polynucleotide of claims 1, 2, 3 or 4 operably associated with a heterologous coding sequence.

11. A transgenic, non-human animal comprising the polynucleotide of claims 1, 2, or 4.

12. The polynucleotide of claim 6, wherein the heterologous coding sequence is a reporter gene.

13. The polynucleotide of claim 12, wherein the reporter gene is luciferase.

14. A method for identifying a test compound capable of modulating prostate-specific gene expression comprising:
(a) measuring the level of expression of a reporter gene under the control of a super-PSA regulatory region, or a transcriptionally active fragment thereof, in the presence and absence of said test compound,
such that if the level obtained in the presence of the test compound differs from that obtained in its absence, then a compound which modulates prostate-specific gene expression is identified.

15. The method of claim 14 wherein the reporter gene is luciferase.

16. A pharmaceutical composition comprising the test compound identified by the method in claim 14.

17. A method for delivery of a therapeutic molecule comprising, introducing into prostate cancer cells of a subject a vector comprising a supre-PSA regulatory region sequence, or transcriptionally active fragment thereof, operatively linked to a heterologous nucleic acid which encodes said therapeutic molecule.

18. A method for inhibiting or treating prostate-related cancer or other proliferative disorder comprising introducing into a cell of a subject a vector comprising a super-PSA regulatory region sequence, or transcriptionally active fragment thereof, operatively linked to a heterologous nucleic acid whose gene product is capable of killing said cell.

19. A method for preventing or delaying a prostate-related disorder comprising introducing into a cell of a subject a vector comprising a super-PSA regulatory region sequence, or transcriptionally active fragment thereof, operatively linked to a heterologous nucleic acid which encodes a therapeutic molecule which is capable of preventing or delaying said disorder.

20. The method of claim 19, wherein said disorder is prostate cancer.


SEQUENCE LISTING (2 sequences)

SEQ ID NO: 1 - DNA, 434 bp, Homo sapiens
ggtgaccagagcagtctaggtggatgctgtgcacacggggtttgtgccactggtgagaaacctgaagattaggaatcctcaatcttatactggacaactgcaagcctgctcagcctttgtctcgatgaagatattatcttcatgatcttggattgaaaacagacctactctggaggaacatattgtatcgattgtcttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggagaattgcctcccaacgctgttcagccagaggcttccacccttgtct

SEQ ID NO: 2 - DNA, 170 bp, Homo sapiens
gctagctctccctccccttccacagctctgggtgtgggagggggttgtccagcctccagcagcatccccagggccttggtcagcctctgggtgccagcagggcaggggcggagtcctggggaatgaaggttttatagggctcctgggggaggctccccagccccaagctt